(19) United States
(12) Patent Application Publication
(10) Pub. No.: US 2013/0240122 A1
(43) Pub. Date: Sep. 19, 2013

(54) METHOD OF MANUFACTURING A PERSONAL HYGIENE PRODUCT

(71) Applicant: NORDSON CORPORATION, Westlake, OH (US)

(72) Inventor: Brian K. Adams, Gainesville, GA (US)

(73) Assignee: NORDSON CORPORATION, Westlake, OH (US)

(21) Appl. No.: 13/787,177

(22) Filed: Mar. 6, 2013

Related U.S. Application Data
(60) Provisional application No. 61/610,063, filed on Mar. 13, 2012.

(51) Int. Cl. A61F 13/15

(52) U.S. Cl. CPC A61F 13/15577
USPC 156/161; 156/60

(57) ABSTRACT

A method of manufacturing a disposable absorbent personal hygiene product including a stretched elastic strand and a flat nonwoven substrate includes mixing a pressurized gas and hot melt adhesive to form a foamed adhesive. A filament of foamed adhesive is discharged toward the stretched elastic strand and deposited onto the stretched elastic strand so that the foamed adhesive expands in volume on the stretched elastic strand. The stretched elastic strand is secured to the flat nonwoven substrate with the foamed adhesive. Alternatively, the foamed adhesive secures two flat nonwoven substrate portions of the disposable absorbent personal hygiene product together.

### CROSS-REFERENCE TO RELATED APPLICATION

This application claims the priority of U.S. Provisional Patent Application Ser. No. 61/610,063, filed on Mar. 13, 2012 (pending), the disclosure of which is incorporated by reference herein.

### TECHNICAL FIELD

This invention relates to dispensing methods for applying foamed hot melt adhesive to various components of a disposable absorbent personal hygiene product during the manufacture of the disposable absorbent personal hygiene product.

### BACKGROUND

Liquid adhesive, such as hot melt adhesive, is applied onto various components during manufacture of a disposable absorbent personal hygiene product such as diapers, adult incontinence products, and feminine hygiene products. Various dispensing systems have been developed for applying hot melt adhesive onto various components of the disposable absorbent personal hygiene product. In one example, these dispensing systems apply a laminating or bonding layer of hot melt adhesive between two flat substrates, such as a nonwoven fibrous layer and a thin polyethylene backsheet. In another example, one or more hot melt adhesive filaments are applied to one or more thin elastic strands and the strand(s) are then adhered to a nonwoven substrate to form an elasticized portion of the disposable absorbent personal hygiene product. Downstream of the dispensing system, the various components (e.g., flat substrate layers and elastic strands) pass through a pressure nip to secure the components together.

In these applications, hot melt adhesive filaments must be carefully controlled during dispensing to ensure that the desired adhesive pattern is accurately applied to the thin elastic strands or within well-defined narrow areas on a flat substrate. In known dispensing systems, continuous filaments of liquid adhesive are discharged from a die or nozzle with one or more adhesive outlets. Each adhesive outlet typically has associated process air outlets that discharge air jets at the dispensed filament. The air jets attenuate each liquid adhesive filament and cause the filaments to move generally in a back and forth manner, a spiral manner, or another manner depending upon the position of the air jets. When the filaments are deposited on a moving flat substrate, the filament forms either an overlapping pattern or a non-overlapping pattern. Consequently, the liquid adhesive filament is carefully controlled for accurate positioning and adherence to the moving strand(s) or flat substrate.

In order to carefully control the liquid adhesive filament, the process air jets used in these known dispensing systems move at a high velocity that is sufficient to modify the flight of the liquid adhesive filaments in a controlled manner. However, the use of relatively high velocity air can result in excessive “fly” in which the filaments are blown away from the desired dispensed pattern. The “fly” results in adhesive deposition outside of the desired boundary of the pattern. Using relatively high velocity air jets can also lead to “shot” in which adjacent adhesive filaments become entangled and form globules of adhesive on the substrate rather than the desired pattern. Consequently, known dispensing systems require the adhesive to have a sufficiently high viscosity and/or density to enable repeatable and accurate control while minimizing “fly” and “shot.”

When evaluating the effectiveness of an adhesive bond between one or more elastic strands and one or more flat substrates, a characteristic that is often measured is creep resistance. “Creep” of an elastic strand is defined as the movement of either end of the elastic strand from an initial location where the end is adhered to a substrate. The level of creep resistance indicates how well the ends of the elastic strand remain adhered in position with respect to a substrate adhered to the elastic strand. Because the elastic strand is adhered to the substrate(s) in a stretched condition, the elastic strand constantly applies force to the substrate and the adhesive in an attempt to return to a relaxed, non-stretched condition. This force enables an elasticized portion of a disposable absorbent personal hygiene product (e.g., leg gathers on a diaper) to remain firmly engaged with the skin surface during use of the product. If an elastic strand in a disposable absorbent personal hygiene product undergoes any significant amount of creep after assembly, at least one end of the elastic strand will effectively de-bond from the substrate and reduce the ability of the elasticized portion to remain firmly engaged with the skin surface. To avoid this undesirable creep, a high quality bond must be formed by the adhesive applied to the elastic strand so that the elastic strand does not de-bond from the substrate.

One well understood method of improving the quality of an adhesive bond, and thereby reducing creep, is by applying additional adhesive on the substrate(s) or the elastic strands. However, applying too much adhesive to the elastic strand locks the elastic strand along its length and thereby reduces the effectiveness of the elastic material to apply force to the substrate. In other words, the elastic strand loses the ability to apply sufficient retraction force to the substrate.

Moreover, increasing the amount of adhesive used in disposable absorbent personal hygiene product manufacturing significantly increases cost and also reduces the “hand” or softness of the resulting product. Applying too much adhesive material increases the stiffness of the resulting product and may also lead to “burn through,” which occurs when the adhesive material burns or melts through the adhered substrate. Especially in diaper manufacturing, the softness of the assembled product is another important measurement used to evaluate the quality of the disposable absorbent personal hygiene product. Consequently, the amount of adhesive used to adhere elastic strands to substrates should be minimized while also maintaining a high level of creep resistance, a high retraction force, and minimized burn through and stiffness. Adhesive dispensing systems should carefully control the discharged liquid adhesive filament to ensure accurate placement of the adhesive material and a high quality bond with minimized use of adhesive.

Also when constructing a disposable absorbent personal hygiene product, two or more substrates may be adhered together by a pattern of adhesive applied to one or both of the substrates. For example, two substrates may be adhered along edge portions of the substrates. As a result, the adhesive filaments discharged towards the substrate(s) must be carefully controlled to ensure accurate positioning along and within the edges of the substrate(s), also referred to as “edge control.” If the adhesive filament undergoes any non-negligible amount of “fly” away from the desired pattern on the substrate(s), then the adhesive is characterized as having poor edge control, which adversely affects the resulting construction of elements in the disposable absorbent personal hygiene product. To this end, adhesive dispensing systems must carefully control the liquid adhesive filament to avoid excessive “fly” and poor edge control in adherence of substrate(s).

By contrast, in other adhesive dispensing fields such as adhesive dispensing on packaging (e.g., boxes), the total amount of adhesive used in an application has been minimized by injecting nitrogen or another gas into the liquid hot melt adhesive to form a foamed adhesive. The gas is injected into liquid adhesive by a foaming mixer that conventionally is a large piece of equipment requiring significant manufacturing space. The foamed adhesive is then deposited in a pattern onto relatively large bonding areas of the packaging. As a result of the large bonding areas used in the packaging fields, highly precise and accurate control of the adhesive is not an important design consideration. More particularly, the foamed adhesive is sprayed, in most circumstances, with a wide pattern that is unassisted by air rather than being discharged as a filament moved by process air. In addition, forming strong bonds between the large bonding areas is not an important design consideration in the packaging field. To this end, even when process air is used with foamed adhesive in these applications, relatively low velocity process air streams may be used to control the flight of foamed adhesive filaments in the packaging field. Thus, any problems of “fly” and “shot” in the packaging field caused by using a low density foamed adhesive are minimized because of the low velocity of the process air.

However, these low velocity process air streams do not adequately control the adhesive filaments when dispensing adhesive onto an elastic strand or onto a nonwoven substrate used in a disposable absorbent personal hygiene product. Furthermore, it was believed that using high velocity process air streams with a lower density adhesive such as foamed adhesive would cause significant “fly” and “shot,” which leads to low creep resistance and/or poor edge control in the disposable absorbent personal hygiene products field. In addition, the large size of conventional foaming mixers prevented manufacturers from positioning the foaming mixers in close proximity to the adhesive applicators, which is desired in nonwoven applications. As a result, foamed adhesive has not been used in the manufacture of disposable absorbent personal hygiene products.

There is a need, therefore, for a method of dispensing adhesive in the manufacture of a disposable absorbent personal hygiene product that provides improved characteristics, including creep resistance, force retraction, and softness in the resulting product.

### SUMMARY

In one embodiment of the invention, a method of manufacturing a disposable absorbent personal hygiene product includes mixing a pressurized gas and hot melt adhesive to form a foamed adhesive. A filament of the foamed adhesive is discharged toward a stretched elastic strand. The filament of foamed adhesive is impacted with high velocity process air to move the filament. The filament of foamed adhesive is deposited onto the stretched elastic strand such that the foamed adhesive expands in volume on the stretched elastic strand. The method also includes securing the stretched elastic strand to a first flat substrate portion with the foamed adhesive.

In one aspect, the process air is a plurality of air jets directed to impart a spiral motion on the filament. Due to the adhesive filament moving in a spiral motion and the elastic strand moving faster than the filament, the filament contacts the stretched elastic strand at first and second contact points and begins to wrap around the stretched elastic strand and stretch between the first and second contact points. In this regard, the stretched elastic strand accelerates the filament of foamed adhesive such that the filament forms localized masses of adhesive at the first and second contact points separated by a thin fiber section that breaks as the adhesive engages the stretched elastic strand. When the thin fiber section breaks, the halves or sections on either side of the break snap back towards the respective first and second contact points and wrap around the stretched elastic strand at those contact points to form the localized masses of adhesive, which are configured to become discrete bond points when securing the stretched elastic strand to the first flat substrate portion. The discrete bond points are separated by sections of stretched elastic strand with no adhesive or minimal adhesive material such that the stretched elastic strand is not rigidly bonded to the first flat substrate portion between the discrete bond points, thereby maximizing the elasticity of the stretched elastic strand in those sections.

The foamed adhesive begins expanding in volume during flight and prior to deposit on the stretched elastic strand. Moreover, the method includes expanding the foamed adhesive in volume by at least 14% total during flight and after deposit on the stretched elastic strand. In another aspect, the process air includes multiple air jets directed in a manner that imparts a substantially back-and-forth motion or any kind of desired motion on the filament. The amount of pressurized gas mixed with a predetermined volume of hot melt adhesive may be increased to increase the amount of foaming that the foamed adhesive will undergo following discharge. This increased foaming leads to increased creep resistance of the stretched elastic strand following securing to the first flat substrate portion. More particularly, the mixing of the pressurized gas and the hot melt adhesive may include sufficient quantities of pressurized gas to result in at least 26% total expansion in volume of the foamed adhesive deposited onto the stretched elastic strand, and preferably enough to result in at least 34% total expansion in volume of the foamed adhesive. This expansion provides a desirable creep resistance, such as less than 10% creep, for industry-standard add on weights.

The method may also include securing a second flat substrate portion to the stretched elastic strand and to the first flat substrate portion with the foamed adhesive. The first and second flat substrate portions may be provided as separate substrates in some embodiments, and may alternatively be provided as separate portions of a single flat substrate (e.g., folded over itself) in other embodiments. In embodiments where the disposable absorbent personal hygiene product includes a plurality of stretched elastic strands, the method includes discharging a plurality of filaments of foamed adhesive and impacting those filaments with process air before deposit onto the plurality of stretched elastic strands.

In another embodiment of the invention, a method of manufacturing a disposable absorbent personal hygiene product includes mixing a pressurized gas and hot melt adhesive to form a foamed adhesive. A filament of the foamed adhesive is discharged toward a first flat nonwoven substrate. The filament of foamed adhesive is impacted with high velocity process air to move the filament. For example, the process air is a plurality of air jets directed asymmetrically towards one another to produce a randomized pattern of adhesive on the nonwoven substrate. Alternatively, the process air is a plurality of air jets that produces a spiral pattern of adhesive on the nonwoven substrate. The filament of foamed adhesive is deposited onto the first flat nonwoven substrate such that the foamed adhesive expands in volume on the first flat nonwoven substrate. The method also includes securing the first flat nonwoven substrate to a second flat nonwoven substrate with the foamed adhesive.

Various additional features and advantages of the invention will become more apparent to those of ordinary skill in the art upon review of the following detailed description of the illustrative embodiments taken in conjunction with the accompanying drawings.

### BRIEF DESCRIPTION OF THE DRAWINGS

FIG. 1 is a schematic perspective view of a disposable absorbent personal hygiene product during assembly of various components.

FIG. 2 is a perspective view of foamed adhesive being applied to an elastic strand.

FIG. 3 is a perspective view of foamed adhesive being applied to a plurality of elastic strands.

FIG. 4 is a schematic diagram view of one embodiment of an adhesive dispensing system used to perform the method of the invention.

FIG. 5 is a cross-sectional side view of an exemplary foaming mixer used in the adhesive dispensing system of FIG. 4.

FIG. 6 is a perspective view of an exemplary adhesive applicator used in the adhesive dispensing system of FIG. 4 to produce a pattern of foamed adhesive on elastic strands as shown in FIG. 3.

FIG. 7 is an enlarged perspective view of the adhesive applicator of FIG. 6.

FIG. 8 is a rear view of the adhesive applicator of FIG. 6, showing internal flow paths for adhesive and process air.

FIG. 9 is a graphical representation showing test results for creep resistance and add-on weight for various levels of foaming the adhesive when using the adhesive dispensing system of FIG. 4.

FIG. 10 is a top view of foamed adhesive being applied to a flat substrate.

FIG. 11 is a first cross-sectional side view taken along line 11-11 in FIG. 13 of another exemplary adhesive applicator used in the adhesive dispensing system of FIG. 4 to produce a pattern of foamed adhesive on a substrate as shown in FIG. 10.

FIG. 12 is a second cross-sectional side view taken along line 12-12 in FIG. 13 of the adhesive applicator of FIG. 11.

FIG. 13 is a bottom view of the adhesive applicator of FIG. 11.

### DETAILED DESCRIPTION OF THE ILLUSTRATIVE EMBODIMENTS

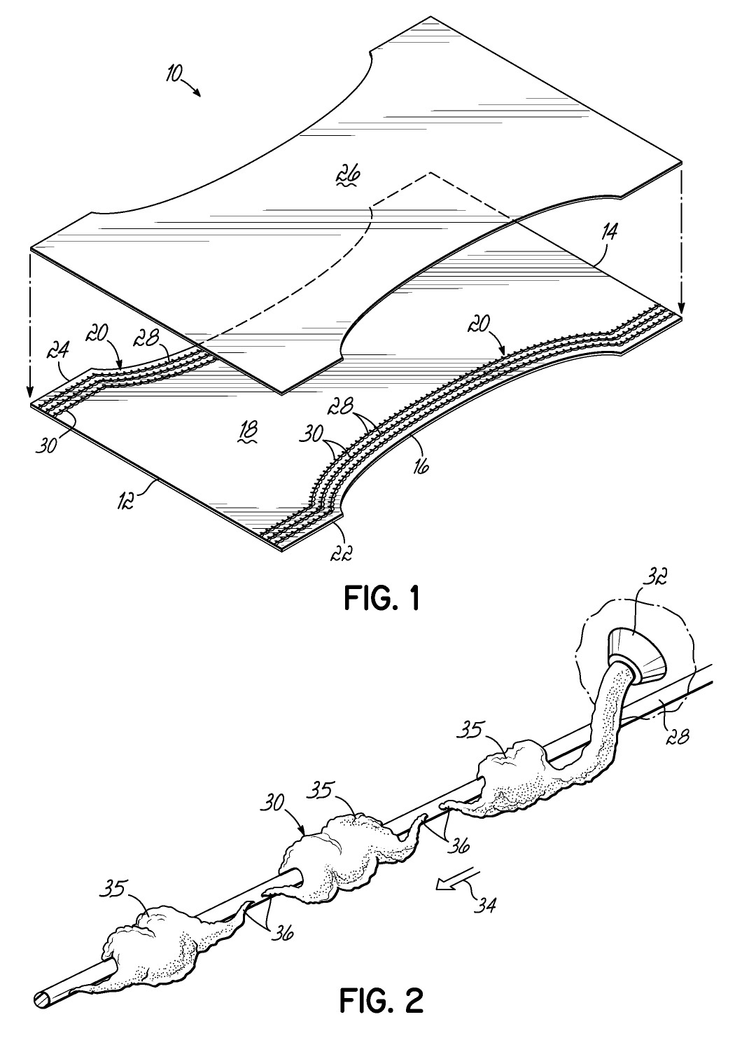

FIG. 1 illustrates one embodiment of a disposable absorbent personal hygiene product 10 manufactured using an illustrative method of the invention. The illustrated disposable absorbent personal hygiene product 10 is a disposable diaper 10 including first and second ends 12, 14 configured to wrap around the waist of the user and a narrowed central portion 16 configured to extend between the legs of the user. The diaper 10 includes a flat nonwoven substrate portion 18 (hereinafter first substrate 18 or flat nonwoven substrate 18), leg gathers 20 formed along each longitudinal side 22, 24 of the diaper 10 between the first and second ends 12, 14, and a second flat substrate portion 26 (hereinafter second flat substrate 26) secured to the nonwoven substrate 18 to enclose the leg gathers 20. The leg gathers 20 are formed by one or more elastic strands 28 that are secured to the nonwoven substrate 18 in a stretched condition so as to provide the diaper 10 with elasticity around the legs of the user. As shown, the second flat substrate 26 is a separate substrate from the flat nonwoven substrate 18 in the illustrated embodiment. However, it will be understood that the second flat substrate 26 alternatively could be a folded-over portion (not shown) of the flat nonwoven substrate 18 in other embodiments. The nonwoven substrate 18, leg gathers 20, and second substrate 26 are secured to each other with hot melt adhesive 30.

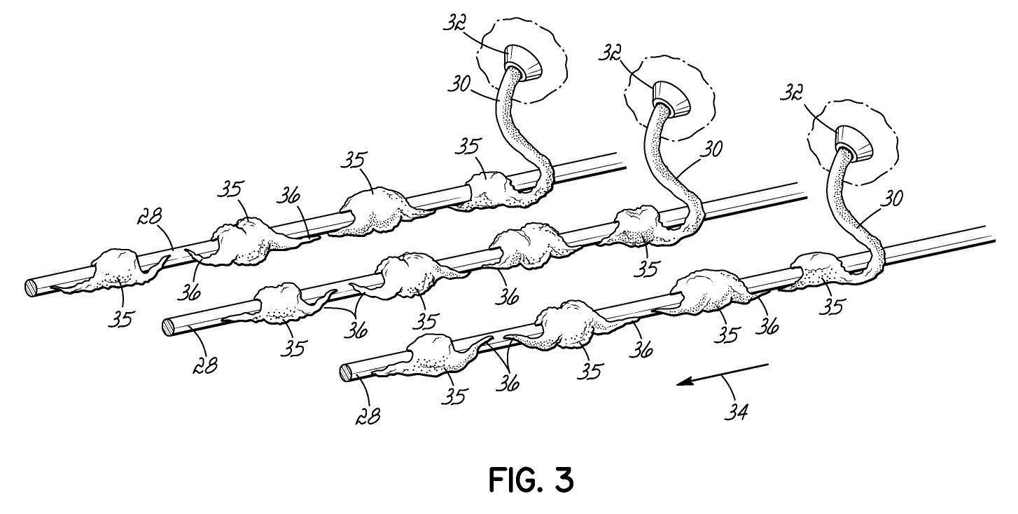

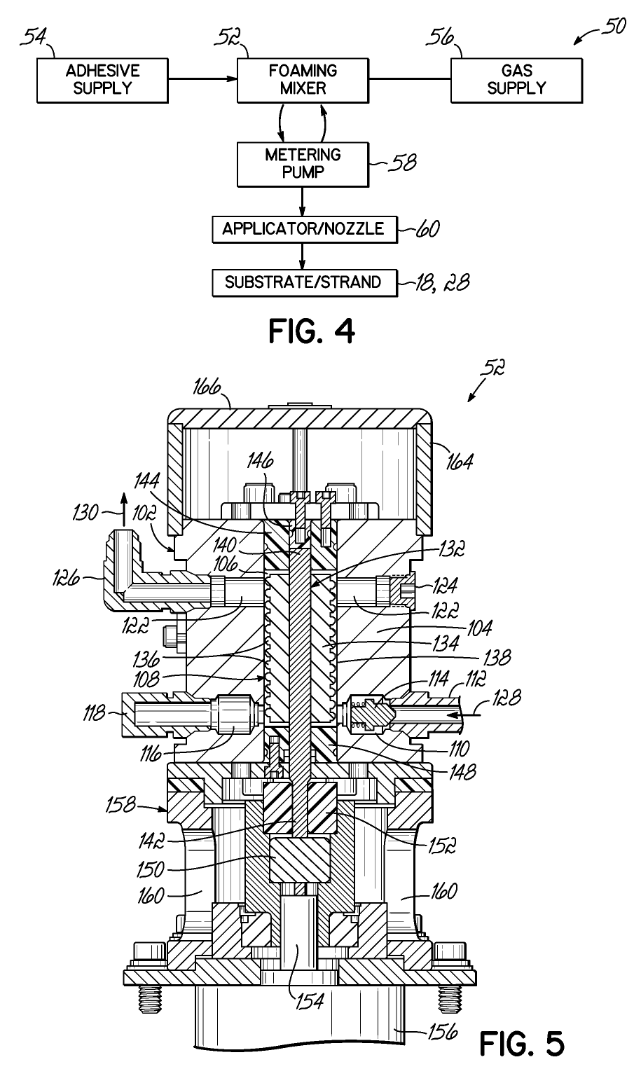

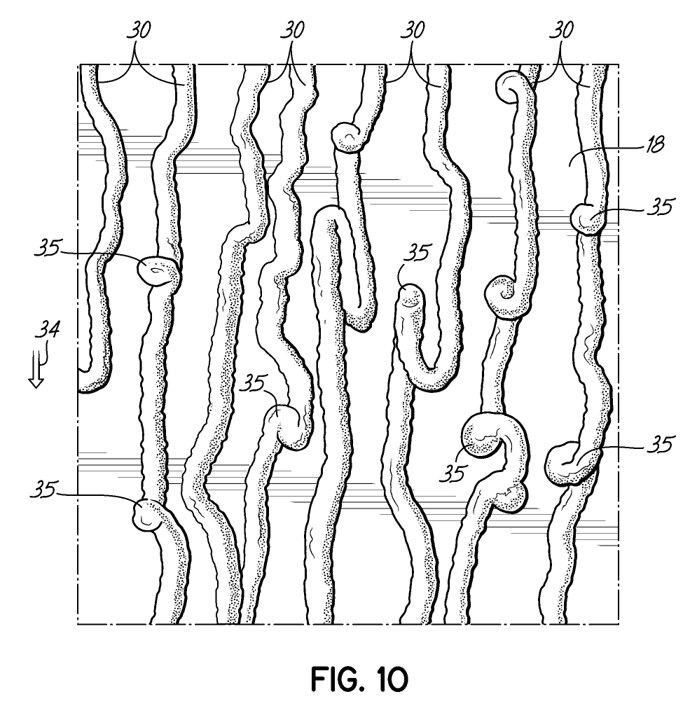

To achieve various benefits in the manufacture and use of the diaper 10, the hot melt adhesive 30 is injected with a pressurized gas such as nitrogen to form a foamed adhesive 30, which is then dispensed on the nonwoven substrate 18 or onto a stretched elastic strand 28. Exemplary deposits of foamed adhesive 30 are shown in FIG. 2 on an elastic strand 28, in FIG. 3 on a plurality of elastic strands 28, and in FIG. 10 on a nonwoven substrate 18. A schematic of an adhesive dispensing system 50 configured to produce these exemplary deposits of foamed adhesive 30 is provided in FIG. 4 and described in further detail below.

With reference to FIG. 2, a filament of the foamed adhesive 30 exits a nozzle outlet 32 in a pressurized state such that the filament of foamed adhesive 30 generally resembles a bead of liquid adhesive immediately after discharge. However, the foamed adhesive 30 begins expanding in volume immediately upon discharge from the nozzle outlet 32 as shown in FIG. 2. The filament of foamed adhesive 30 is impacted by air jets to cause the filament of foamed adhesive 30 to move with a generally spiral motion and wrap around the elastic strand 28, which is moving in the direction of arrow 34. As shown in FIG. 2, the foamed adhesive 30 is accelerated and stretched out when applied to the faster moving elastic strand 28, which causes the foamed adhesive 30 to form a plurality of localized areas of increased masses 35 of adhesive coupled by thinner filament sections 36 of adhesive. In the preferred operation, these thinner filament sections 36 will break between adjacent masses 35, leaving no adhesive between the localized adhesive masses 35. These localized adhesive masses 35 become discrete bond points during bonding of the elastic strand 28 to the substrate 18, which advantageously provides desirable force retraction and creep resistance qualities.

The expansion in volume of the foamed adhesive 30 occurs both before and after deposit of the filament of foamed adhesive 30 on the elastic strand 28. The volume of the filament of foamed adhesive 30 increases by up to 100% total (for example), after discharge from the nozzle outlet 32 as a result of the expansion of nitrogen within the hot melt adhesive 30. In this regard, the filament of foamed adhesive 30 is up to 50% less dense than a liquid bead of adhesive having a similar volume. It will be understood that the filament of foamed adhesive 30 could be deposited onto a plurality of parallel stretched elastic strands 28 (i.e., at least two elastic strands 28) in other embodiments of the invention, and the claims of this application are thus not limited to deposit of a single filament of foamed adhesive 30 onto a single elastic strand 28. It will also be understood that depositing the foamed adhesive 30 onto the elastic strand 28 may result in the foamed adhesive 30 being in substantially complete contact the with elastic strand 28 as shown in FIG. 2, but this wrap or deposit onto the elastic strand 28 may also result in the foamed adhesive 30 only coming into partial contact with the elastic strand 28 with another portion of the foamed adhesive 30 drooping away from the elastic strand 28 in other embodiments.

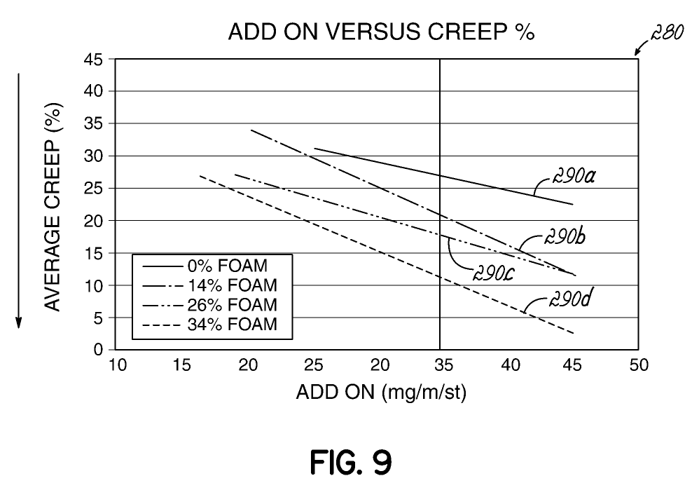

With reference to FIG. 3, the foamed adhesive 30 may be deposited onto a plurality of parallel elastic strands 28 moving along the direction indicated by arrow 34. Similar to the deposit of foamed adhesive 30 onto a single elastic strand 28 as described above, each filament of foamed adhesive 30 is discharged from a corresponding nozzle outlet 32 and begins expanding in volume immediately upon discharge. Once again, each filament of foamed adhesive 30 is accelerated upon deposit onto the respective elastic strand 28 such that the foamed adhesive 30 stretches out to form a plurality of localized areas of increased masses 35 of adhesive separated by broken-apart filament sections. These adhesive masses 35 advantageously form discrete bond points for each elastic strand 28 when adhered to the substrate 18. It will be understood that a single filament of foamed adhesive 30 may be moved with a swirl motion across multiple elastic strands 28 to form these discrete bond points in other embodiments consistent with this invention. In addition to reducing the total volume of adhesive 30 being applied to the elastic strand 28, the foamed adhesive 30 unexpectedly exhibits improved creep resistance compared to liquid adhesive as evidenced in test results discussed with reference to FIG. 9 below. Accordingly, the dispensing system and methods of the current application significantly reduce adhesive add on and manufacturing costs while improving and/or maintaining the bond quality necessary in the disposable absorbent personal hygiene product field.

As briefly discussed above, FIG. 4 shows an exemplary embodiment of an adhesive dispensing system 50 used to dispense one or more filaments of foamed adhesive 30. The adhesive dispensing system 50 may include components such as the Signature® spray nozzles and SureWrap® nozzles commercially available from Nordson Corporation of Westlake, Ohio, but it will be understood that any type of spray nozzle may be used without departing from the scope of the invention. The adhesive dispensing system 50 includes a foaming mixer 52 connected to an adhesive supply 54 and a pressurized gas supply 56. The foaming mixer 52 is operable to mix or inject pressurized gas into hot melt adhesive. The foaming mixer 52 is coupled to a metering pump 58 and an adhesive dispenser or applicator 60. The metering pump 58 supplies a metered amount of foamed adhesive 30 to the applicator 60 while recycling the remaining foamed adhesive 30 back to the foaming mixer 52. Thus, the foamed adhesive 30 in the metering pump 58 remains pressurized and continuously replenished by the foaming mixer 52. The applicator 60 discharges the foamed adhesive 30 and process air to produce a desired deposit of foamed adhesive 30 onto either a nonwoven substrate 18 or an elastic strand 28 as described above with reference to FIGS. 2 and 3.

In one embodiment, the foaming mixer 52 is a mixer as described in U.S. Pat. No. 7,703,705 to Ganzer, the entire disclosure of which is hereby incorporated by reference herein. This exemplary embodiment of a foaming mixer 52 is also shown in FIG. 5. The foaming mixer 52 includes a mixing body 102 with a side wall 104, a tubular mixing chamber 106 bound by the side wall 104, and a mixer element 108 located inside the mixing chamber 106. The mixer 52 is heated by heaters (not shown), such as cartridge-style resistance heating elements, embedded in the side wall 104. The heaters are controlled using feedback from a conventional temperature sensor (not shown), such as a resistance temperature detector, a thermistor or a thermocouple. The heaters ensure that the adhesive material in the mixer 52 is maintained within an acceptable temperature range for dispensing by the applicator 60.

In one example, the foaming mixer 52 receives Bostik 2861 hot melt adhesive and nitrogen supplied at about 50 psi. The foaming mixer 52 is maintained at about 315 degrees Fahrenheit and operates at about 600 rpm such that the foamed adhesive 30 exits the foaming mixer 52 at a pressure of about 900 psi. With these settings, the foaming mixer 52 is operative to discharge about 20-45 milligrams per meter of elastic strand 28 when the adhesive dispensing system 50 applies adhesive to an elastic strand 28.

The foaming mixer 52 includes an adhesive inlet port 110 leading into the mixing chamber 106. The adhesive supply 54 is coupled in fluid communication with the adhesive inlet port 110 by an elbow fitting 112 mounted in the adhesive inlet port 110 by, for example, a threaded engagement and a supply hose (not shown) connected to the elbow fitting 112. A flow control element such as a spring-loaded check valve 114 is located in the adhesive inlet port 110 between the mixing chamber 106 and the adhesive supply 54. The check valve 114, which has a conventional construction, prevents gas-filled adhesive material from infiltrating into the elbow fitting 112 and being transported upstream to the adhesive supply 54.

The foaming mixer 52 also includes a gas inlet port (not shown) leading into the mixing chamber 106. The gas supply 56 is coupled in fluid communication with the gas inlet port. The gas inlet port may be disposed adjacent to the adhesive inlet port 110 in the mixing body 102. The foaming mixer 52 also includes a measurement port 116 extending into the mixing chamber 106 opposite the adhesive inlet port 110. The measurement port 116 receives an elbow connector 118 configured to receive a pressure gage (not shown) for measuring the pressure within the mixing chamber 106. The foaming mixer 52 includes a pair of outlet ports 122 extending into the mixing chamber 106. One of the outlet ports 122 is inactive in FIG. 5 and closed with a plug 124, while the other outlet port 122 receives an elbow fitting 126 leading to the metering pump 58 and the applicator 60. Consequently, adhesive material enters the foaming mixer 52 at the adhesive inlet port 110 as shown by arrow 128 and leaves the foaming mixer 52 at the outlet port 122 as shown by arrow 130.

The mixer element 108 includes a central shaft 132 extending longitudinally through the mixing chamber 106, a cylindrical body 134 rigidly coupled for rotation with the central shaft 132, and fins 136 that project outwardly from the cylindrical body 134 toward the confronting inner surface 138 of the side wall 104 of the mixing chamber 106. The central shaft 132 includes a first end 140 located adjacent to the outlet ports 122 and a second end 142 located adjacent to the adhesive inlet port 110. The first end 140 of the central shaft 132 is supported for rotation relative to the side wall 104 by a bushing or bearing 144 in the mixing chamber 106. A thrust bearing 146 fitted in the bushing 144 provides a thrust load support for central shaft 132. The bushing 144 and the thrust bearing 146 are assembled together and secured to the mixing body 102 with conventional threaded fasteners.

The second end 142 of the central shaft 132 projects through another bushing 148 situated in the mixing chamber 106 adjacent the adhesive inlet port 110. Another thrust bearing 150 provides a thrust load support for the second end 142 of the central shaft 132. The second end 142 of the central shaft 132 is coupled by a coupling element 152 with a drive shaft 154 of a motor 156. The coupling element 152 and the thrust bearing 150 adjacent the coupling element 152 are formed from a material having a low thermal conductivity so that heat transfer is reduced from the mixing body 102 to the motor 156. The motor 156 is also isolated thermally from the mixing body 102 by a standoff 158 separating the motor 156 from the mixing body 102. The standoff 158 includes slots 160 that promote cooling. The motor 156 drives the powered rotation of the drive shaft 154 and the central shaft 132 for moving the fins 136 relative to the side wall 104 of the mixing chamber 106.

The adhesive material is bounded inside the mixing chamber 106 in a region between the bushings 144, 148. The bushings 144, 148 include various sealing members that assist in confining the fluid material inside the mixing chamber 106. A cowling 164 and a cap 166 are secured by conventional fasteners to the mixing body 102 and protectively cover the mixing body 102 opposite the motor 156.

The fins 136 on the mixer element 108 are distributed in rows along the length of the cylindrical body 134 (and the length of the central shaft 132). The tip of each fin 136 has a close clearance with the side wall 104. The adhesive material and the pressurized gas delivered into the mixing chamber 106 are forced through gaps between adjacent fins 136, as the fins 136 rotate, for mixing, stirring and agitating the gas and adhesive material into the foamed adhesive 30. Rotation of the fins 136 relative to the stationary side wall 104 therefore operates to repeatedly divide the gas and adhesive into small streams and then recombine the streams to create a substantially homogeneous blend or mixture of gas and adhesive with the pressurized gas entrained in solution.

The fins 136, which are fashioned from an initially continuous helical thread extending along the length of the cylindrical body 134, define a helical arrangement likewise winding along the length of the cylindrical body 134. As the central shaft 132 of the mixer element 108 is continuously rotated by operation of motor 156, the helical arrangement of the fins 136 tends to force the adhesive material toward the adhesive inlet port 110, which counters the forward flow of the gas/adhesive mixture toward the outlet ports 122. The foaming mixer 52 is therefore operable to supply foamed adhesive 30 to the metering pump 58 and the applicator 60. Additionally, the foaming mixer 52 is smaller in size than many conventional foaming mixers, which enables the foaming mixer 52 to be positioned in close proximity to the metering pump 58 and the applicator 60.

The metering pump 58 may be the pump used with the VersaBlue® melters commercially available from Nordson Corporation of Westlake, Ohio. The metering pump 58 operates at a rotational speed similar to the rotational speeds used during metering of liquid adhesive because the pressurized foamed adhesive 30 in the metering pump 58 is in substantially a liquid state. For example, the foamed adhesive 30 may circulate between the foaming mixer 52 and the metering pump 58 at about 900 psi to maintain the substantially liquid state until discharge from the applicator 60. The metering pump 58 then delivers a metered supply of the foamed adhesive 30 into one of the applicators 60 described below. It will be understood that different types of pumps may be used in other embodiments.

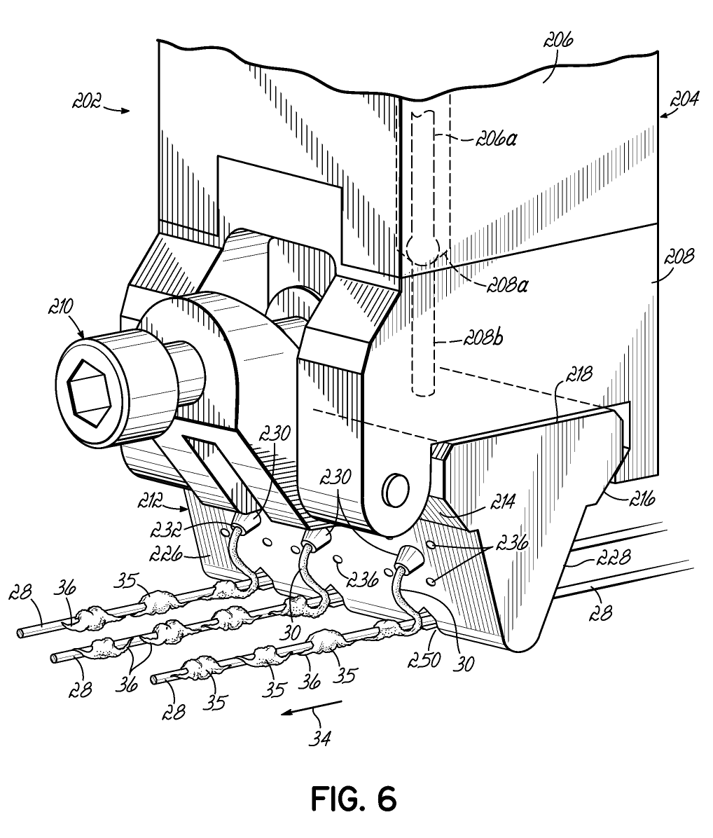

In one embodiment, the applicator 60 is a spiral dispensing module 202 as described in U.S. Pat. No. 7,578,882 to Harris et al., the entire disclosure of which is hereby incorporated by reference herein. This embodiment of a dispensing module 202 is also shown in FIGS. 6-8.

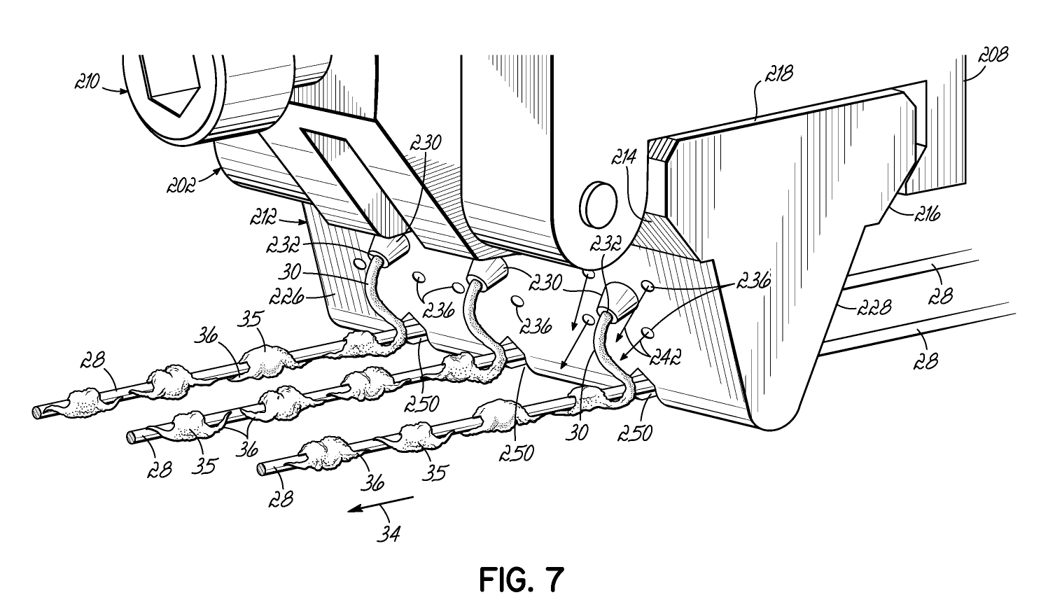

With reference to FIGS. 6 and 7, the dispensing module 202 includes a module body 204 including a central body portion 206 and a lower body portion 208. The module 202 also includes a clamping or quick disconnect mechanism 210 for connecting a nozzle 212 to the lower body portion 208. As well understood, the central body portion 206 may include a valve stem 206a that engages with a valve seat 208a formed in the lower body portion 208 to control flow of hot melt adhesive into a passage 208b leading to the nozzle 212 (the valve stem and valve seat may also be located in other locations within the dispensing module 202 in other embodiments). The quick disconnect mechanism 210 is further described in U.S. Pat. No. 6,619,566 to Gressett, Jr. et al., the entire disclosure of which is hereby incorporated by reference herein. The nozzle 212 receives pressurized foamed adhesive 30 and pressurized process air from respective supply passages (208b, not shown) located in the lower body portion 208.

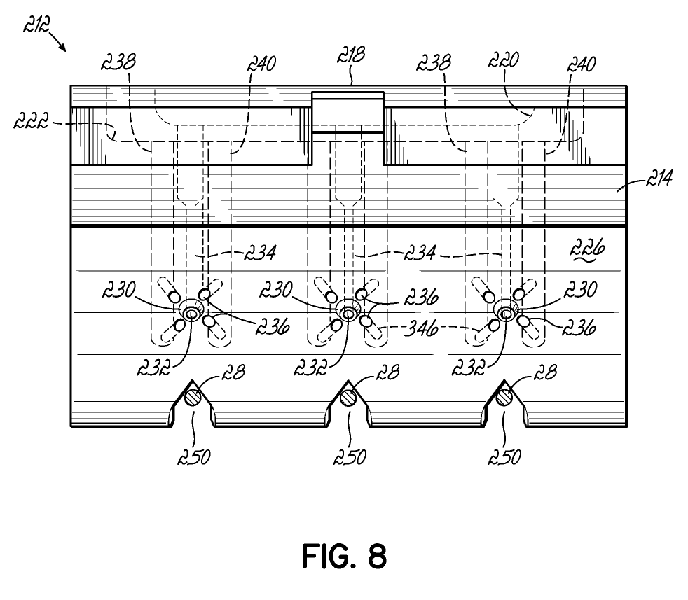

Referring now to FIGS. 7 and 8, the exemplary nozzle 212 is shown in more detail. The nozzle 212 includes angled cam surfaces 214, 216, as more fully described in U.S. Pat. No. 6,619,566, to facilitate coupling the nozzle 212 with the dispensing module 202. The nozzle 212 includes a first side 218 configured to mount to the lower portion 208 of the dispensing module 202. The first side 218 includes an adhesive supply port 220 and at least one process air supply port 222 which mate to the corresponding adhesive and air supply passages in the dispensing module 202. The nozzle 212 defines a generally wedge-shaped cross-section including second and third sides 226, 228. A plurality of frustoconically-shaped protrusions 230 extend from the second side 226 of the nozzle 212, each including an adhesive discharge outlet 232 disposed on a distal end of the protrusion 230. The adhesive discharge outlets 232 are in fluid communication with adhesive discharge passages 234, which in turn are in communication with the adhesive supply port 220. At least a portion of the adhesive discharge passages 234 are oriented to form an acute angle with a plane parallel to the first side 218, and thus form an angle with a direction corresponding to of movement of the strand 28, generally indicated by arrow 34. The adhesive discharge passages 234 of the exemplary embodiment are inclined at approximately 20° to the first side 218, whereby the foamed adhesive 30 is dispensed from the adhesive discharge outlets 232 to the elastic strands 28 generally in the direction of strand movement.

The second side 226 of the nozzle 212 further includes a plurality of air discharge outlets 236 proximate each of the adhesive discharge outlets 232 and in fluid communication with air discharge passages 238, 240, which are in communication with the air supply port 222 on the first side 218 of the nozzle 212. In the exemplary nozzle 212, four air discharge outlets 236 are disposed in a generally square pattern around each adhesive discharge outlet 232 at the base of the frustoconical protrusion 230. The air discharge passages 238, 240 of the exemplary nozzle 212 are angled with respect to the corresponding adhesive discharge passage 234 so that high velocity process air jets indicated by arrows 242 are directed to be tangential to a discharged filament of foamed adhesive 30 from the adhesive discharge outlet 232. Each air discharge outlet 236 is positioned at the same radial distance from a common center defined at the location of the corresponding adhesive discharge outlet 232. Consequently the process air jets tangentially swirl about the discharged filament of foamed adhesive 30 at generally the same location downstream of the adhesive discharge outlet 236 and the air discharge outlets 236. Variation of the filament movement pattern is possible by adjusting the offset spacing and orientation of the air discharge passages 238, 240 relative to the adhesive discharge passage 234, as will be apparent to those skilled in the art. In one alternative, the process air includes at least two air jets directed in a manner that imparts a substantially back-and-forth motion on the filament.

The nozzle 212 further includes notches 250 formed into an end of the nozzle 212 opposite the first side 218 and proximate the adhesive discharge outlet 232 to direct the elastic strands 28 past the air and adhesive discharge outlets 232, 236 disposed on the second side 226 of the nozzle 212. As shown more clearly in FIG. 8, the notches 250 extend between the second and third sides 226, 228 of the nozzle 212. The notches 250 guide each elastic strand 28 to be located below the corresponding adhesive discharge outlet 232. In an exemplary embodiment, the second and third sides 226, 228 are configured to form acute angles with the first side 218. In one exemplary embodiment, the second side 226 forms an angle of approximately 60°-80° with the first side 218. In another aspect of the invention, the third side 228 forms an angle no greater than approximately 70° with the first side 218. Advantageously, the angle of the third side 228 facilitates the passage of knots formed in the elastic strand 28 without causing breakage of the elastic strand 28.

In operation, an elastic strand 28 is received into each notch 250 and moves in a direction indicated by the arrow 34. As the elastic strands 28 pass beneath the adhesive discharge outlets 232, a filament of foamed adhesive 30 is dispensed from each adhesive discharge outlet 232, generally toward the corresponding elastic strand 28 so as to be deposited at least partially on the elastic strand 28. More specifically, the filament of foamed adhesive 30 may be deposited in complete contact with the elastic strand 28, or may be in partial contact with other portions of the foamed adhesive 30 drooping from the elastic strand 28. Pressurized process air is discharged from the air discharge outlets 236 and directed generally tangentially toward the filaments of foamed adhesive 30, as depicted by arrows 242. The pressurized process air causes the filaments of foamed adhesive 30 to move in a spiral motion as the filaments are deposited on the elastic strands 28. As described in greater detail above, the elastic strands 28 accelerate the filaments 30 and cause the filaments 30 to stretch and form discrete masses 35 of adhesive that form discrete bond points when the elastic strands 28 are adhered to a substrate 18. The filaments of foamed adhesive 30 expands in volume on the elastic strands 28 as shown in FIGS. 6 and 7. The elastic strands 28 are then ready to be secured to another substrate in the disposable absorbent personal hygiene product 10, such as to form the leg gathers 20 of a diaper 10.

As described above, the foamed adhesive 30 is highly pressurized and maintained in nearly liquid form until discharge from the applicator 60. Thus, the foamed adhesive 30 is not completely expanded in volume when process air impacts or tangentially contacts or otherwise moves the discharged filaments of foamed adhesive 30 in flight. As a result, the density of the foamed adhesive 30 remains high enough to avoid fly or shot caused by the high pressure of the process air. Moreover, even though the foamed adhesive 30 begins expanding prior to deposit on an elastic strand, the foamed adhesive 30 retains enough integrity in flight to avoid bouncing off the elastic strand. The filaments of foamed adhesive 30 are still adequately and precisely controllable so as to be deposited in desired patterns on the elastic strand(s) 28, similar to filaments of liquid hot melt adhesive.

When using the foamed adhesive 30 to bond one or more elastic strands 28 to a nonwoven substrate 18, the bond quality exhibited by the foamed adhesive 30 is substantially similar to the bond quality formed by liquid adhesive. For example, using the same volume of foamed adhesive 30 and liquid adhesive results in substantially similar levels of creep resistance. Furthermore, the foamed adhesive 30 continues to form discrete bond points along the elastic strand 28 during bonding, which provides high force retraction qualities. Considering that the foamed adhesive 30 includes about half of the normal amount of hot melt adhesive material as a liquid adhesive, the resulting softness or hand of the diaper 10 is improved compared to conventional designs. The expansion of the foamed adhesive 30 effectively forms a web-like structure of hot melt adhesive and gas that effectively adheres to the elastic strand 28 upon deposit on the elastic strand 28.

In another related example, foaming of the foamed adhesive 30, to a larger extent, provides improved creep resistance for the same amount of add on adhesive weight. With reference to FIG. 9, test results are shown that prove the amount of creep resistance has been increased by the application of more foaming to the adhesive in operation. In this regard, FIG. 9 includes a graph 280 representing average creep in percent measured against add on adhesive weight in milligrams per meter of elastic strand 28. The creep measurements were taken after an extended aging period of 28 hours following bonding of the elastic strand 28 to a nonwoven substrate 18, and various data points (not shown) were plotted on the graph 280 for different levels of foaming (0% or liquid adhesive, 14%, 26%, and 34%) within the foamed adhesive 30. By “% foaming,” the percentages shown in this Figure mean the percent of total volume expansion undergone by the foamed adhesive during and after deposit onto the elastic strand 28. Although the industry standard add on range is about 35-50 milligrams per meter, test results were provided both inside and outside this standard range so that trend lines 290a, 290b, 290c, 290d could be generated to illustrate the improvements for different levels of foaming the foamed adhesive 30.

As shown in the graph 280, the addition of more foaming to the foamed adhesive 30 significantly improved the resulting creep resistance over the extended sample aging period of 28 hours. To this end, the first trend line 290a for liquid adhesive (0% foam) shows a relatively high creep of about 25% for the industry standard add on range, while the second trend line 290b for 14% foaming drops the creep exhibited down toward 15% in the industry standard add on range. The third trend line 290c for 26% foaming further reduces the amount of creep exhibited in the test results, and the fourth trend line 290d for 34% foaming achieves an ideal amount of creep (e.g., less than 10%) within the industry standard add on range. Therefore, providing enough pressurized gas in the foamed adhesive 30 to result in at least 26% foaming or total volume expansion, or even more preferably, at least 34% foaming, provides a desirable level of creep resistance for most applications in the industry-standard add on range for adhesive weight per length of strand. It will be understood that the specific percentages of foaming and levels of creep resistance achieved may vary based on material differences for some adhesives in other embodiments. However, for all materials tested, increasing the amount of foaming that occurs by increasing the amount of pressurized gas entrained within the liquid hot melt adhesive will result in improved creep resistance for the same amount of add on weight. Thus, foaming the foamed adhesive 30 provides unexpected benefits in improving creep resistance while maintaining a high bond quality.

Furthermore, the expansion of the foamed adhesive 30 results in more rapid cooling of the outermost or external layers of adhesive material, and thus reduces the likelihood of the foamed adhesive 30 burning through a second substrate 26. More specifically, the foamed adhesive remains warm enough to form a reliable adhesive bond between elements of the diaper 10 while cooling enough to avoid burn-through on temperature sensitive substrates. Additionally, testing has revealed the unexpected benefit that the use of foamed adhesive 30 reduces the pinch pressure that a pressure nip or pressure roller needs to apply to produce the high quality bond between the elastic strands 28 and the substrate 18. Thus, less forceful pressure nips may be utilized with the adhesive dispensing system 50 of this invention.

Turning to another embodiment shown in FIG. 10, a plurality of filaments of foamed adhesive 30 exit corresponding nozzle outlets (not shown) in a pressurized state so that the filaments of foamed adhesive 30 resemble beads of liquid adhesive upon initial discharge from the nozzle outlets. Process air in the form of multiple air jets is also discharged and moves the filaments of foamed adhesive 30 in a desired manner before deposit on the nonwoven substrate 18. The substrate is moving in the direction of arrow 34. The process air is operative to cause any desired motion of the filaments, including but not limited to, randomized motion or spiral motion. In this embodiment, the process air imparts a randomized motion of each filament of foamed adhesive 30 as evidenced by the random pattern of adhesive on the nonwoven substrate 18. As shown in FIG. 10, the foamed adhesive 30 forms a number of localized areas of increased or overlapping adhesive masses 35 along the length of the substrate 18. These localized adhesive masses 35 become discrete bond points during bonding of an elastic strand 28 to the substrate 18, which advantageously provides adequate force retraction and creep resistance qualities of the resulting bond. Similar to the elastic strand embodiment shown in FIGS. 2 and 3, the foamed adhesive 30 begins expanding in volume immediately after discharge from the nozzle outlet and increases in volume by at least 14% total during flight and after deposit on the nonwoven substrate 18. Furthermore, the foamed adhesive 30 produces adequate full coverage of the substrate 18 with less add on and a thinner overall adhesive coating.

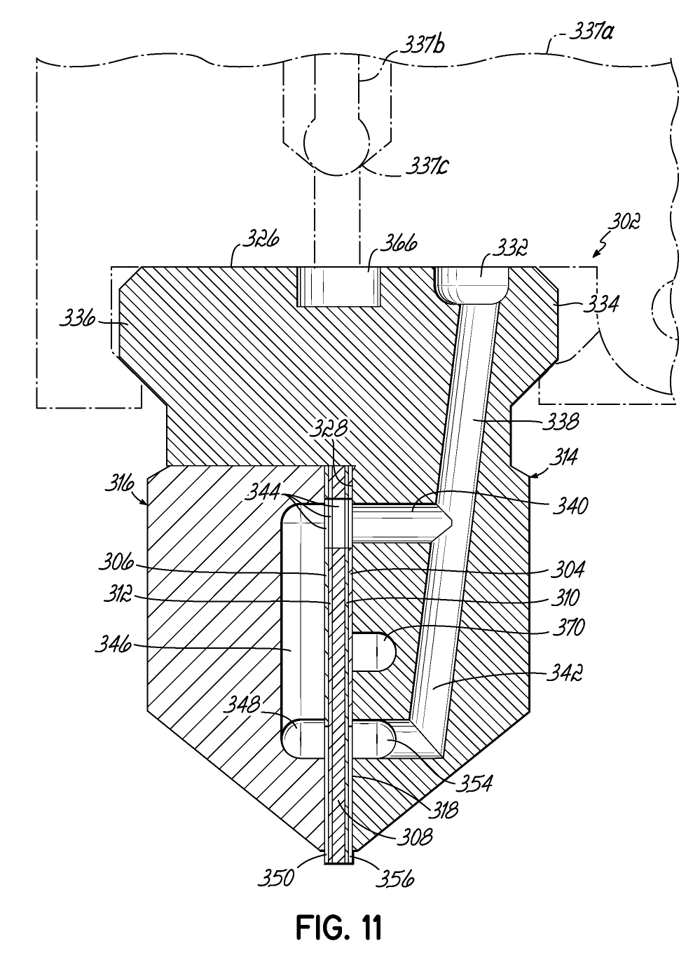

In another embodiment, the applicator 60 includes a dispensing nozzle 302 as described in U.S. Patent Publication No. 2010/0327074 to Bondeson et al., the entire disclosure of which is hereby incorporated by reference herein. This exemplary embodiment of a dispensing nozzle 302 is also shown in FIGS. 11-13.

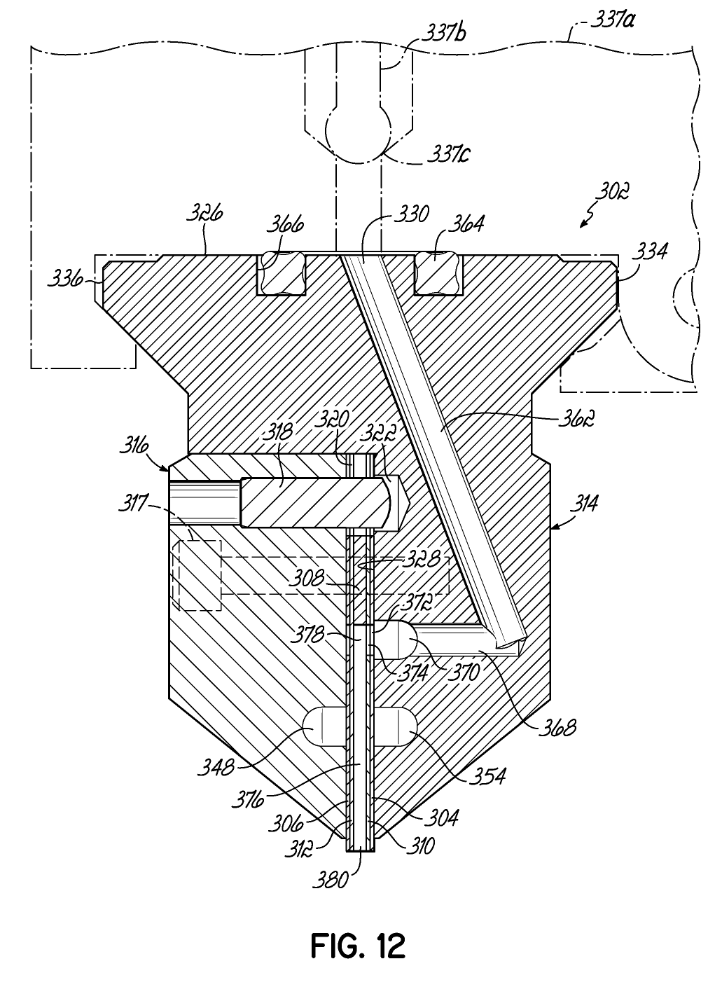

With reference to FIGS. 11 and 12, the nozzle 302 includes first and second process air shim plates 304, 306, an adhesive shim plate 308, first and second separating shim plates 310, 312, and first and second end plates 314, 316. The entire assembly of plates 304, 306, 308, 310, 312, 314, 316 is held together by a pair of threaded fasteners 317 that extend through corresponding apertures in the first end plate 314 (as well as the shim plates 304, 306, 308, 310, 312) and into threaded holes in the second end plate 316. The shim plates 304, 306, 308, 310, 312 are arranged in order from the first end plate 314 to the second end plate 316 as follows: the first process air shim plate 304 (adjacent the first end plate 314), the first separating shim plate 310, the adhesive shim plate 308, the second separating shim plate 312, and the second process air shim plate 306 (adjacent the second end plate 316). The second end plate 316 also includes a projection 318 serving as a locating member that extends through respective upper slots 320 in the air shim plates 304, 306, the separating shim plates 310, 312, and the adhesive shim plate 308. The projection 318 is then received in a blind bore 322 in the first end plate 314.

The first end plate 314 is a generally L-shaped member and includes a top surface 326 generally orthogonal to planes that contain the first and second process air shim plates 304, 306, the adhesive shim plate 308 and the first and second separating shim plates 310, 312. A side surface 328 generally parallel to the planes containing these same shim plates 304, 306, 308, 310, 312 receives the threaded fasteners. The top surface 326 includes an adhesive inlet 330 and a process air inlet 332. The first end plate 314 also includes oppositely extending projections 334, 336 at the top surface 326 that may be used for securing the nozzle 302 to a dispensing valve or module 337a as well understood. The dispensing module 337a includes a valve stem 337b that may engage with a valve seat 337c to control flow of hot melt adhesive into the adhesive inlet 330 of the nozzle 302.

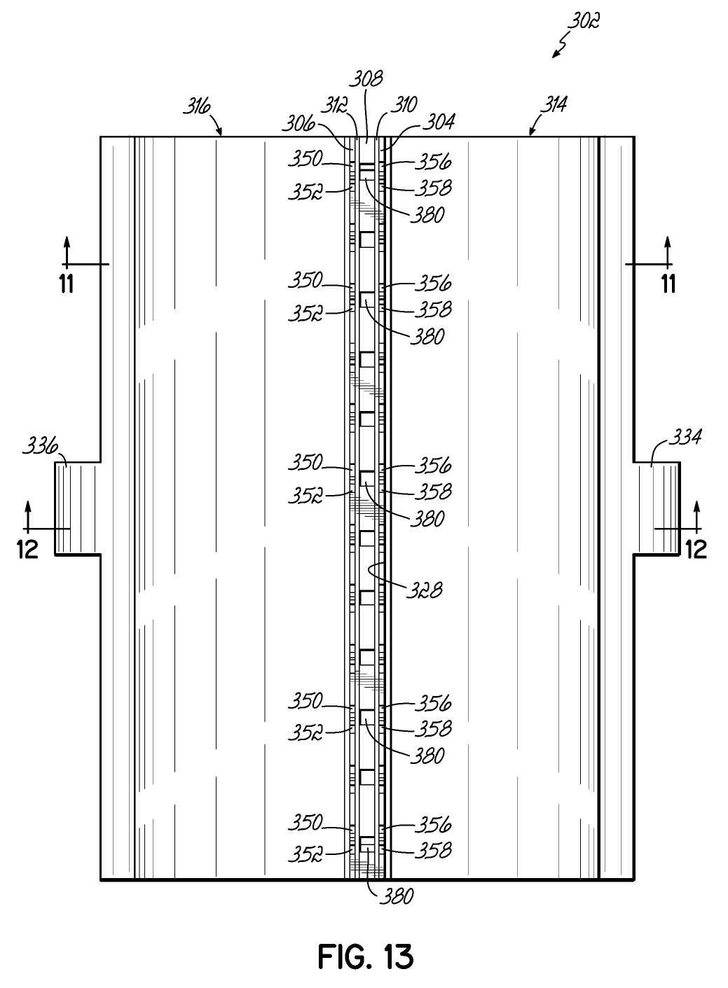

With reference to FIG. 11, the first end plate 314 includes a process air inlet passage 338 communicating with the process air inlet 332. The process air inlet passage 338 communicates with first and second air distribution passages 340, 342 that respectively communicate with opposite sides of the shim plate assembly 304, 306, 308, 310, 312. The first air distribution passage 340 passes through the shim plate assembly 304, 306, 308, 310, 312 at aligned holes 344, then through a vertical recess 346 in the second end plate 316, and finally into a horizontally extending slot 348 in the second end plate 316. Process air from the first air distribution passage 340 enters corresponding pairs of air slots 350, 352 (FIG. 13) in the second process air shim plate 306. The second air distribution passage 342 extends into a horizontally extending recess 354 in the first end plate 314. Process air from the second air distribution passage 342 enters corresponding pairs of air slots 356, 358 (FIG. 13) in the first process air shim plate 304. The arrangement of the air slots 350, 352, 356, 358 is described in further detail with reference to FIG. 13 below.

With reference to FIG. 12, the first end plate 314 includes an adhesive inlet passage 362 communicating with the adhesive inlet 330. A seal member 364 located in a groove 366 may be used to seal the adhesive inlet 330 at the top surface 326. The adhesive inlet passage 362 communicates with an adhesive distribution passage 368 and an upper horizontal slot 370 in the first end plate 314. This upper horizontal slot 370 communicates with the adhesive shim plate 308 via respective aligned apertures 372, 374 in the first process air shim plate 304 and the first separating shim plate 310. The adhesive shim plate 308 includes a plurality of adhesive slots 376 each having an adhesive inlet 378 and an adhesive outlet 380. It will be understood that the second process air shim plate 306 also includes an adhesive aperture 372 to allow full interchangeability between the first and second process air shim plates 304, 306. However, the adhesive aperture 372 in the second process air shim plate 306 is blocked from use by the second separating shim plate 312 in the embodiment shown in FIG. 12. The separating shim plates 310, 312 are utilized to seal off the respective air slots 350, 352, 356, 358 from the adhesive slots 376.

Each of the adhesive slots 376 is located generally in the center of a corresponding set of air slots 350, 352, 356, 358 in the first and second process air shim plates 304, 306. Thus, as shown in FIG. 13, each pair of air slots 350, 352 in the second process air shim plate 306 is directly aligned with a corresponding pair of air slots 356, 358 in the first process air shim plate 304, so as to surround a corresponding adhesive outlet 380 in the adhesive shim plate 308. Although not clearly shown in FIG. 13, the air slots 350, 352 converge toward each other along their length and the air slots 356, 358 converge toward each other along their length such that the process air ejected from each pair of air slots 350, 352, 356, 358 is configured to intersect. However, none of the air slots 350, 352, 356, 358 converge toward their associated adhesive outlet 380 because the respective pairs of slots 350, 352, 356, 358 are each contained in parallel planes different from the plane containing the adhesive slot 376.

In operation, pressurized high velocity process air is delivered into the nozzle 302 at the process air inlet 332 and then is discharged from the corresponding air slots 350, 352, 356, 358 in the first and second process air shim plates 304, 306. The pressurized foamed adhesive 30 is delivered into the nozzle 302 at the adhesive inlet 330 and then is discharged from the corresponding adhesive slots 376 at the adhesive outlets 380. It will be understood that any number of adhesive slots 376 and adhesive outlets 380 may be provided along the length of the nozzle 302 depending on the substrate to be coated with adhesive 30.

The discharged stream of pressurized air exiting from each air slot 350 converges and impacts against a process air stream exiting from each associated air slot 352. In a similar manner, respective process air streams exiting air slots 356 impact against the streams exiting from air slots 358. These asymmetric impacts cause the filaments of foamed adhesive 30 exiting the associated adhesive outlets 380 to move side-to-side or back and forth in random directions. As a result, the filaments of foamed adhesive 30 form an erratic, non-uniform or random pattern as, for example, shown in FIG. 10. However, this pattern of adhesive 30 exhibits good edge control on the substrate 18 because excessive “fly” or “shot” is avoided during the dispensing process. As previously described with reference to FIG. 10, the nozzle 302 is therefore operable to deposit a random pattern of filaments of foamed adhesive 30, the foamed adhesive 30 increasing in volume on the nonwoven substrate 18. Additionally, testing has revealed the unexpected benefit that the foamed adhesive 30 permeates more readily into the relatively porous nonwoven material of the substrate 18 than a corresponding liquid bead of adhesive. As a result, less total adhesive add on is required to form an adequate bond with the nonwoven substrate 18. In sum, the foamed adhesive 30 produces high quality bonds with good edge control and substantially less adhesive add on than what is present when using liquid adhesive.

While the present invention has been illustrated by the description of specific embodiments thereof, and while the embodiments have been described in considerable detail, it is not intended to restrict or in any way limit the scope of the appended claims to such detail. The various features discussed herein may be used alone or in any combination. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope or spirit of the general inventive concept. What is claimed is:

1. A method of manufacturing a disposable absorbent personal hygiene product including a stretched elastic strand and a first flat substrate portion, the method comprising:
mixing a pressurized gas and hot melt adhesive to form a foamed adhesive;
discharging a filament of the foamed adhesive toward the stretched elastic strand;
impacting the discharged filament of foamed adhesive with process air to move the filament of foamed adhesive;
depositing the filament of foamed adhesive onto the stretched elastic strand;
expanding the foamed adhesive in volume during flight and after deposit onto the stretched elastic strand; and
securing the stretched elastic strand to the first flat substrate portion with the foamed adhesive.

2. The method of claim 1, wherein impacting the discharged filament of foamed adhesive with process air further comprises:
impacting the discharged filament of foamed adhesive with multiple air jets directed to impart a spiral motion to the filament during flight.

3. The method of claim 2, wherein depositing the filament of foamed adhesive onto the stretched elastic strand further comprises:
stretching the filament of foamed adhesive during deposit on the stretched elastic strand such that the filament forms localized masses of adhesive configured to become discrete bond points when securing the stretched elastic strand to the first flat substrate portion.

4. The method of claim 1, wherein impacting the discharged filament of foamed adhesive with process air further comprises:
impacting the discharged filament of foamed adhesive with multiple air jets directed to impart a back-and-forth motion to the filament during flight

5. (canceled)

6. The method of claim 1, wherein mixing the pressurized gas and hot melt adhesive further comprises:
mixing a sufficient quantity of pressurized gas with the hot melt adhesive to result in at least 14% total expansion in volume of the foamed adhesive deposited onto the stretched elastic strand.

7. The method of claim 1, wherein mixing the pressurized gas and hot melt adhesive further comprises:
increasing an quantity of pressurized gas that is mixed with a predetermined volume of hot melt adhesive to increase the amount of total expansion in volume that the foamed adhesive will undergo following discharge, thereby increasing creep resistance of the stretched elastic strand after securing to the first flat substrate portion.

8. The method of claim 6, wherein mixing the pressurized gas and hot melt adhesive further comprises:
mixing a sufficient quantity of pressurized gas with the hot melt adhesive to result in at least 26% total expansion in volume of the foamed adhesive deposited onto the stretched elastic strand.

9. The method of claim 8, wherein mixing the pressurized gas and hot melt adhesive further comprises:
mixing a sufficient quantity of pressurized gas with the hot melt adhesive to result in at least 34% total expansion in volume of the foamed adhesive deposited onto the stretched elastic strand.

10. The method of claim 1, further comprising:
securing a second flat substrate portion to the stretched elastic strand and to the first flat substrate portion with the foamed adhesive to enclose the stretched elastic strand.

11. The method of claim 10, wherein the first and second flat substrate portions include separate first and second substrates, and the method further comprises:
enclosing the stretched elastic strand between the first and second substrates.

12. The method of claim 10, wherein the first and second flat substrate portions are a single flat substrate, and the method further comprises:
folding a first section of the single flat substrate over the stretched elastic strand and a second section of the single flat substrate; and
enclosing the stretched elastic strand between the first and second sections of the single flat substrate.

13. The method of claim 1, wherein the disposable absorbent personal hygiene product includes a plurality of stretched elastic strands, and the method comprises:
discharging a plurality of filaments of the foamed adhesive toward the plurality of stretched elastic strands;
impacting the plurality of discharged filaments of foamed adhesive with process air to move the filaments of foamed adhesive;
depositing each of the plurality of filaments of foamed adhesive onto a corresponding one of the plurality of stretched elastic strands such that the foamed adhesive expands in volume on the stretched elastic strands; and
securing the plurality of stretched elastic strands to the first flat substrate portion with the foamed adhesive.

14. A method of manufacturing a disposable absorbent personal hygiene product including first and second flat nonwoven substrates, the method comprising:
mixing a pressurized gas and hot melt adhesive to form a foamed adhesive;
discharging a filament of the foamed adhesive toward the first flat nonwoven substrate;
impacting the discharged filament of foamed adhesive with process air;
depositing the filament of foamed adhesive onto the first flat nonwoven substrate;
expanding the foamed adhesive in volume during flight and after deposit onto the first flat nonwoven substrate; and
securing the first flat nonwoven substrate to the second flat nonwoven substrate with the foamed adhesive.

15. The method of claim 14, further comprising:
impacting the discharged filament of foamed adhesive with process air to produce a random adhesive pattern deposited on the first flat nonwoven substrate.

16. The method of claim 14, further comprising:
impacting the discharged filament of foamed adhesive with process air to produce a spiral adhesive pattern deposited on the first flat nonwoven substrate

17. (canceled)

18. The method of claim 14, wherein mixing the pressurized gas and hot melt adhesive further comprises:
mixing a sufficient quantity of pressurized gas with the hot melt adhesive to result in at least 14% total expansion in volume of the foamed adhesive deposited onto the first flat nonwoven substrate.

19. The method of claim 14, further comprising:
discharging a plurality of filaments of the foamed adhesive toward the first flat nonwoven substrate;
impacting the discharged plurality of filaments of foamed adhesive with process air;
depositing the plurality of filaments of foamed adhesive onto the first flat nonwoven substrate such that the foamed adhesive expands in volume on the first flat nonwoven substrate.
